(19) United States
(12) Patent Application Publication
(10) Pub. No.: US 2016/0144152 A1
(43) Pub. Date: May 26, 2016

(54) POSITIONING SYSTEM FOR A MEDICAL DEVICE

(71) Applicant: Sayed Nour, Chaville (FR)

(72) Inventor: Sayed Nour, Chaville (FR)

(21) Appl. No.: 14/897,985

(22) PCT Filed: Jun. 6, 2014
(86) PCT No.: PCT/EP2014/061917

§ 371 Date: Dec. 11, 2015

Related U.S. Application Data
(60) Provisional application No. 61/834,132, filed on Jun. 12, 2013.

(51) Int. Cl. A61M 25/01; A61N 1/362; A61N 1/05; A61M 16/04; A61J 15/00

(52) U.S. Cl. CPC A61M 25/0105; A61M 16/0447; A61M 16/0465; A61M 16/0488; A61J 15/0003; A61N 1/05; A61N 1/362; A61B 2034/2048

(57) ABSTRACT

A positioning system is disclosed comprising at least three elements, wherein the first element is adapted to be attached to said invasive device, the second element is adapted to be attached to a predefined surface area of a body, and the third element is adapted to be connected to a displaying means. The first element and the second element create a signal, to be received by the third element and which is indicative of the position of the first element within the body. The third element is adapted to receive and visualize said signal emitted from the at least one first and second elements.

The present invention pertains to a system for facilitating the introduction in and monitoring the position of an invasive device within a body. In particular, a positioning system is disclosed comprising at least three elements, wherein the first element is adapted to be attached to said invasive device, the second element is adapted to be attached to a predefined surface area of a body, and the third element is adapted to be connected to a displaying means. The first element and the second element create a signal, to be received by the third element and which is indicative of the position of the first element within the body. The third element is adapted to receive and visualize said signal(s) emitted from the at least one first and second elements.

Emergency situations or even life-threatening situations, such as sudden cardiac arrest (SCA), require quick aid to the inflicted person. First aid often needs to be performed by a bystander, which is not medically trained, and may encompass measures such as mouth-to-mouth breathing and cardiac massage followed by informing a medical practitioner.

Mouth-to-mouth ventilation is first priority procedure in Out-of-Hospital Cardiac Arrest (e.g. drowning), with its efficacy remaining debated due to insufficient tidal volume, interruption of cardiac massage, associated iatrogenic trauma, incidences of regurgitation, etc. In addition, the efficacy of the laryngeal mask airway (LMA) application during CPR does not benefit the respiratory status of patients according to recent multi-centers study population.

Adequate measures may, however, also require advanced actions like tracheal intubation, tracheostomy or Cricothyroidotomy as well as drugs administrations. Unfortunately, these procedures are hazardous and remain to be performed by medical doctors and not by paramedics, non-medical staff, such as lifeguards, or even passerby's.

Even in case a trained non-medical rescuer is available, a considerable lapse of precious time occurs until the arrival of a complete medical team.

Accordingly, an objective of the present invention resides in the provision of a system and method allowing a non-medical rescuer, such as a paramedic, or an untrained by-stander to perform advanced medical actions such as endotracheal intubation.

Another objective of the present invention resides in the provision of a system and method allowing monitoring the position of an invasive device within a body, wherein the required components are simple to use and easy to transport.

Another objective of the present invention resides in the provision of a system and method avoiding Iatrogenic Traumatic Corporeal Disasters (ITCD), as a consequence of ignorance of medical knowledge, e.g. the human anatomy, physiopathology, etc.

The present positioning system and method address the above-mentioned objectives. The positioning system comprises a first element adapted to be attached to an invasive device, a second element adapted to be attached to a predefined surface area of a body; and a third element adapted to receive and compute the signal(s) received from the first and second elements. The third element is capable to compute the signal(s) received such that the position of the medical device is visualized on a display associated with the third element, so that the user is capable to perceive the position of the medical device in the individually body into which the medical device has been placed in.

All of the above first, second and third elements may be comprised either individually or altogether by more than one entity. I.e. the first, second and/or third element may be comprised by one, two, three, four five or more entities, which may fulfill the same function, that is the respective elements may be constituted by at least one element, i.e. one, two, three four, five or more elements (an element comprises a number of elements all fulfilling the same function), or may be constituted by entities, that together with one, two or more of the other entities of the respective element fulfill the particular function as such (multicomponent element).

The method for employing the present positioning system comprises using a device to which the first element has been attached, attaching the second element to the predefined surface area of a body, connecting said elements with a third element, thus that a position signal is created indicative for the position of said first element within the body, and visualizing the position signal on a display comprised by the third element.

The present system and method allow even an untrained user to perform essentially advanced medical interventions, even those comprising invasive measures, guided directly and/or indirectly by professional medical doctors and systems.

It is conceivable that the present system and method find entrance in households in connection with e.g. stomach tube and manual illustrating the use of the stomach tube and containing information about appropriate rinsing fluids. In case of acute intoxications, e.g. upon ingestion of a poisonous plant, a member of the household may initiate a treatment with or without consulting a physician who may follow the intervention by accessing the information displayed on an electronic device, which may even be a the hand-held electronic device. The present system may be cost effectively produced and is easy to transport and to use.

It is conceivable that the present system and method find entrance in households in connection with e.g. vascular catheter and a manual illustrating the use of the catheter and containing information about appropriate anatomical insertion guidelines. In case of emergency, e.g. SCA with difficulty to find vascular approach due to collapsed venous or arterial systems, the device could be guided and introduce safely for: delivery of pharmacological supports and/or IV fluids. In the same manner pacemaker wires may be introduced through the same technique directly to the right atrioventricular cavities to trigger and maintain heartbeat if needed. Procedures that could be achieved by a member of the household may initiate a treatment with or without consulting a physician who may follow the intervention by accessing the information displayed on an electronic device, which may even be a the hand-held electronic device. The present system may be cost effectively produced and is easy to transport and to use.

Upon intubation, for instance, the rescuer, hereinafter also denominated user, may either use an intubation tube, which is already configured to comprise the first element at a defined location thereof, either integrally or as a separate piece, which is fixedly attached thereto. Alternatively, the user may attach the first element to a defined location of the intubation tube. In a next step, a second element may be attached to a first predefined surface area of the body. The third element, which is either attached to a hand-held electronic device or part thereof, may serve in this case as a second element as well and needs to be attached to a second predefined surface area of the body. Attachment of the second and third elements at a first and second predefined surface area of the human body may be achieved e.g. by attaching the second and third elements to a collar structure, which may be sleeved on the neck of the human body at a defined position, e.g. directly above the larynx, thereby allocating the second element directly above the larynx and the third element in a defined and affixed relationship to the second element.

According to an embodiment the person at site may choose a particular body area from software preinstalled in/on a device associated with the third element, for example the respiratory system, e.g. for endotracheal intubation, the gastric tract or the cardiovascular system, for e.g. an venous or arterial approach. The person may further position the second element near the chosen entrance site of the invasive device, while positioning a further second element at the destination, e.g. at the midsternal wall if cardiac catheterization is requested; at the epigastric area if gastric tube are needed etc. In this way the user at site may easily follow and especially correct the path of the first element by direct communication between the other elements, by vocal and/or mapping system at the screen.

The first element emits a signal, capable of passing through biological tissue, and which is detected by the second and third elements. A position signal indicative for the position of the first element is created either in the third element or in the hand-held electronic device and overlaid on a three dimensional image of the body and displayed to the user. This configuration allows the user to distinguish if the intubation tube passes the opening of the trachea or esophagus. It will be appreciated that the positioning of another second element at a third predefined surface area of the human body enables a spatial position signal. The attachment of more second elements at other predefined surface areas of the human body may improve accuracy of the positioning signal.

According to the present invention, a positioning system comprises a first element adapted to be attached to a medical device conceived for invasive measures, at second element adapted to be attached to a predefined surface area of a body and a third element adapted to be connected to a hand-held electronic device, wherein said first element and said second element are adapted to create a signal indicative of the position of said first element within the body, wherein said third element is adapted to receive and visualize said position signal on said hand-held electronic device.

The first element or first elements is/are either integrally formed with the medical device or is adapted for attachment to the medical device.

It will be appreciated that the first element(s) should be located either at the forward end of the medical device to be inserted, i.e. the tip or close to it, to enable an accurate image presentation of progression of said invasive device within the body. Alternatively the first element(s) may be located at a defined position on the invasive device which is more distant from the tip of said device. In this case, the position signal needs to be subjected to a calculation step before displaying the same to display the tip of the invasive device on the hand-held electronic device. Also two or more first elements may be located on the invasive medical device, either at the tip/top and/or at a location downstream, which provides both, accurate follow up of the progression of said invasive device within the body and a visualization of the position of the device downstream, i.e. line with the human organ/tissue or inclined etc.

Attachment of the first element(s) may be obtained by any means, preferably via an attachment surface, adapted to receive the first element. The attachment surface may be a clip for connecting to a tip area of the invasive device. Alternatively, the first element(s) may be glued to the tip area of the invasive device. It will be appreciated that any physiologically stable and biocompatible glue may be employed. Alternatively a fiberoptic spot may integrated in the corpus of the respective invasive medical device, which could be conventional or not, optionally with an external cover.

The first element(s) may further comprise a transmitter which transfers a continuous or pulse like signal to be received by the second element(s). There are no particular limitations to the nature of the signal. It will be appreciated that said signal should pass through biological tissue, i.e. the body, preferably the human body, and should have sufficient strength to be received by the second element(s), such as e.g. Bluetooth etc. The transmitter may be powered via the invasive device, e.g. by a power supply line located in the invasive device. Alternatively, the transmitter may have an independent power supply by providing the first element(s) with an energy source. A lithium ion battery may serve as a suitable energy source, which is connected to the transmitter

The second element(s) is/are adapted to be located and/or attached to a predefined surface area of a body and to receive the signal from the first element(s). The second element(s) has/have a receiver receiving the signal from the first element(s). The second element(s) further has/have a transmitter to transmit a signal to the third element(s). The receiver and transmitter of the second element(s) may be powered by an independent power supply, for instance a lithium ion battery connected to the receiver and transmitter, respectively. In this case, transmitting of the signal to the third element(s) is preferably performed by wireless transmission.

Since the second element(s) is/are located at a predefined surface area of a body, power supply may be also obtained by establishing a cable connection between the second element(s) and a remote power supply. The hand-held electronic device may serve as the remote power supply. In this case, transmitting of the signal to the third element(s) is preferably performed by a cable transmission.

The second element(s) and optionally further the third element(s) may be attached to a collar or arm cuff, which in turn is attached to a defined position on the body. Alternatively, the second element(s) and optionally further the third element(s) may be attached directly to a predefined surface area of the body. The predefined surface area of the body may depend on the intended use of the invasive device. Correct placement of an endotracheal tube requires for instance certainty that the endotracheal tube is inserted in fact in the trachea and not in the esophagus. In contrast thereto, correct placement of a stomach tube needs information that the stomach tube is inserted in fact in the esophagus and not in the trachea. This may be achieved by attaching the two required or optionally more elements via a collar, which may surround the neck directly above the larynx, and/or near a final destination, e.g. at the epigastric area. One of the elements may be placed on the collar at a position corresponding to a position directly above the larynx. Other elements on the collar have then a defined position with respect to the before mentioned element. It will be appreciated that other clothing or parts thereof may be employed, wherein the clothing or parts thereof comprise second element(s) and optionally further the third element(s) located therein/thereon. Alternatively, said second element(s) and optionally further the third element(s) may be attached to different clothing or parts thereof. Another example of clothing like structure to allocate the elements at predefined positions on the surface of a human body is disclosed in U.S. Ser. No. 13/685,537 assigned to the present inventor and the contents of which are disclosed by way of reference in their entirety. Specifically, U.S. Ser. No. 13/685,537 discloses a medical device close fitting to a patient's body and which may be used for SCA (sudden cardiac arrest) patients. Said medical device may be provided with second element(s) and optionally the third element(s) at defined locations thereof.

Direct attachment of an element (the second element, the third element) may be obtained by adhesion, e.g. by employing a glue. The glue is preferably biocompatible with the human skin and may be easily removed after using the system or method of the present invention.

Preferably, the second element, which may be composed of one, two or more pieces, stays mobile at the patient's body in a matter to guide the position of first element, i.e. the invasive device, until arriving at its final destination. For example in case of endotracheal intubation, one piece will be located externally at the manubrium grove and may other behind the neck, then to be followed downward to the upper chest wall to follow the endotracheal tube downward until before the tracheal bifurcation. Or, further following the introduction of bronchial tube aspiration procedure. Another example, in case of venous or arterial catheterization, one of the second element piece could be mobilized and follow externally at the patient body, by the therapist in a matter to guide the catheter passway until final destination.

The way to the final destination will always be guided and marked externally by second element(s).

The third element(s), are adapted to correct the whole procedure according to captured signals from other elements: First element represented by the invasive device and second element(s), represented by the external guide piece(s).

Attachment of the second and/or third elements at a first and second predefined surface area of the human body may be achieved e.g. by attaching the second and/or third elements to a collar structure, which may be sleeved on the neck of the human body at a defined position, e.g. directly above the larynx, thereby allocating the second element(s) directly above the larynx and the third element(s) in a defined and affixed relationship to the second element

The position of said first element(s) may be calculated via triangulation. Triangulation is well known to the skilled person as well as required calculation steps and signal conversions, if required. Triangulation requires at least two receivers located at different predefined positions (here surface areas of a body) and which receivers receive the signal transmitted from the first element(s). One of said receivers may be located in a second element. The other receiver may be located in another second element, or alternatively in a third element. It will be appreciated that employing two receivers will result in a two-dimensional information content only. Information about the third-dimension may be derived and calculated from the image of the body. Inserting an endotracheal tube, for instance, may require only information if said tube is correctly inserted in the trachea or incorrectly in the esophagus. Accordingly, a two-dimensional information content of the signal is sufficient. A position signal having a three-dimensional/ spatial information content needs at least another second element. Employing additional second elements further enables a more accurate positioning of the first element. Preferably two, three, four, five, six, seven, eight, nine second elements and more preferably ten second elements are employed. It will, however, be clear that more second elements may be employed. It will be also appreciated that each of said second elements needs to be at attached to different predefined surface areas of a body.

Signals from the second element(s) are transmitted to the third element(s). The third element(s) may have a receiver for this purpose. Alternatively, signals from the second element(s) may be transmitted via a cable connection to the third element(s). Said signals are subjected to a triangulation calculation and are optionally subjected to mathematical transformations to obtain a position signal of the first element(s) with respect to the image of the body shown on the display of the hand-held electronic device. Mathematical calculation and transformation steps may be either performed by a processor contained in the third element, wherein the processor is adapted to perform the required calculation steps. Alternatively, the processor of the hand-held electronic device performs the required mathematical calculation and transformation steps.

The skilled person is well acquainted with the construction of the present elements, particular, regarding the transmitters, receivers, energy sources, attachment of the elements to various places, as well as obtaining a position signal and indicating the same on the display of the hand-held electronic device in a manner that the position of the first element(s) is shown in a three-dimensional image of the body.

The term “invasive device” designates any device/tool which is adapted for insertion in a body. The invasive device may be any medical invasive device or a medical surgical device

Preferably, said medical invasive/surgical device is an endotracheal tube, tracheostomy tube, stomach tube or cricothyrotomy tube. Other examples of invasive devices are well known in the art.

According to a preferred embodiment, said first element(s) comprise(s) a transmitter, an energy source and an attachment surface. The transmitter needs to transmit a signal to the second element(s) and optionally to the third element(s) which are located on a surface area of the body. Hence, the transmitter needs to generate a signal which may pass through the biological tissue. In case the body is a human body, the transmitter may generate an infrared signal. The energy source may be a lithium ion battery or any other kind of battery connected to the transmitter of the first element. Preferably, powering of the transmitter may be switched on and off. More preferably, powering may be performed remotely employing the hand-held electronic device. For this purpose, the third element(s) requires a transmitter transmitting a respective on/off signal to a receiver located in the first element.

According to another preferred embodiment, said second element(s) comprises a receiver and a transmitter. The receiver receives the signal from the at least one first element(s) in a wireless manner, e.g. the receiver is an infrared receiver. The transmitter transmits the received signal to the third element. Transmission of the received signal may be performed wireless, e.g. by infrared or any electromagnetic radiation, such as Bluetooth, or tethered by cables.

According to still another preferred embodiment, said third element(s) comprise(s) a receiver and connecting means for connecting said third element(s) to said hand-held electronic device. The receiver of the third element(s) receives the signals from the second element(s) and/or the first element, in case the third element(s) is/are located at a predefined surface area of the body. The received signals may either be processed to a positioning signal by processing means of the third element. Alternatively, the received signals may be transmitted to the hand-held electronic device and processed to a position signal by employing the device's processor. The connecting means for connecting said third element(s) to said hand-held electronic device are preferably conventional connection means employing common data interfaces present at hand-held electronic devices, such as USB, mini- or micro-USB, RS-232, Firewire, SCSI, AGP, PCI.

According to a preferred embodiment, said third element(s) comprise(s) a transmitter. The transmitter of the third element(s) may be used either to transmit received signals to a remote hand-held electronic device for indicating the position of the first element(s) with respect to an image of the body. Alternatively, the transmitter of the third element(s) may be adapted to transmit an on/off signal to the first element(s) and/or the second element(s) to enable simultaneous switching on/off of the present positioning system.

According to another preferred embodiment, said transmitter of said first element(s) and each receiver of said second element(s) communicate via an infrared signal, i.e. infrared radiation.

Infrared radiation is suitable to transmit a signal across the human or animal body tissue with minor signal loss and signal disturbance avoiding at the same time damages to the tissue. IR A radiation is preferred. More preferred are radio waves.

According to still another preferred embodiment, each transmitter of said second element(s) and said receiver of said third element(s) communicate via wireless connection selected from the group consisting of infrared and electromagnetic connection means or wired connection.

According to a preferred embodiment, said hand-held electronic device is selected from the group consisting of a conventional smart phone, notebook and tablet computer. The term “hand-held electronic device” encompasses devices of the before mentioned kind having a weight of five kilograms or less.

According to another preferred embodiment, said hand-held electronic device displays said position signal in a three dimensional illustration of said body. It will be appreciated that three dimensional issues may be presented on a normal display e.g. by employing depth information and image contrast. The three dimensional illustration of said body is preferably a three dimensional illustration of the human body is well known in the art and may e.g. be derived from the web page http://www.visiblebody.com/index.html.

According to still another preferred embodiment, said first element(s) comprise(s) an acceleration sensor. The acceleration sensor may provide additional acceleration data which may be transmitted by a transmitter of the first element(s). The acceleration data may be employed to provide a more accurate orientation and movement of the first element(s) displayed on the hand-held electronic device.

According to a preferred embodiment, said invasive device is an endotracheal tube, tracheostomy tube, stomach tube, cricothyrotomy tube, oropharyngeal tube, laryngeal mask, cardiac pacing wire or a venous, arterial or cardiac catheter. The skilled person is well acquainted with the above mentioned and other invasive devices. Suitable attachment points for attaching the first element(s) to the invasive device as well as suitable attachment means are known to the skilled person.

According to another preferred embodiment, said body is an animal or human body.

According to still another preferred embodiment, said position signal is visualized in real-time. The expression “real-time” as used herein refers to an indication of the actual position of the first element(s) on the hand-held electronic device within a period of less than one second. Preferably, a period of less than a half second, more preferable a period of less than 100 milliseconds is encompassed. Real-time visualization has the further advantage that positioning of the invasive device within the body may not only be timely followed up by the device's user but also by a medical professional participating the display of the hand-held electronic device.

According to a preferred embodiment, the present invention encompasses a method for employing the present system as described above. Said method comprises the steps of attaching said first element(s) to an invasive device, attaching second element(s) to a predefined surface area of a body, connecting third element(s) with a hand-held electronic device, creating a positioning signal indicative of the position of said first element(s) within the body and visualizing the position signal on the hand-held electronic device.

According to another preferred embodiment, the present system may be used for avoiding

Iatrogenic Traumatic Corporeal Disasters (ITCD). ITCD is directed to any inadvertent adverse effect of complication as a result from a medical treatment, such as injuries or trauma arising from incorrect positioning of invasive medical devices.

The proposed technique presents a cost-effective therapeutic method that renders unnecessary the current expensive Cath-lab equipment with hazardous risks of patients as well as therapeutic staff radiations.

The invasive device, which could be in the form of tube (endotracheal, gastric, etc.,) or vascular catheter that may include or not a balloon device as described for example in US2011021987, or pacing wire; and may include several embodiments as been previously explained in US2013018302 such as:
Extra lumen(s), located at the tip of the device for the injections of local anaesthetic (e.g. Xylocaine) drugs during skin insertion or to avoid pharyngeal reflex;
Aspirations of fluids (tracheal, gastric, cardiac tamponade, etc.;
Sensors which could provide direct biochemical and blood-gas analysis;
Malleable guide-wire, to facilitate the introduction of the device in the body. The guide-wire system, could be provided with an integrated cutting head system for facilitating a percutaneous insertion; and/or a blunt tip (e.g. tube).

The screen receiver device, may include software system to trigger patients pulsations, blood gas and biochemical analyses; commanding console (e.g. pacemaker, pulsatile catheter driving system, etc.),

1. Positioning system, comprising:
a first element attached to a medical device for insertion in a body and capable of emitting signals;
a second element attached to a predefined surface area of a body and capable of emitting signals;
a third element connected to an electrical device and comprising display means and being capable to receive and compute signals from the at least one of the first and second elements,
wherein said third element is adapted to visualize said signals on a hand-held electronic device.

2. The system of claim 1, wherein said first, second and/or third element comprise at least two elements each.

3. The system of claim 1, wherein the first element comprises a transmitter, an energy source and an attachment surface.

4. The system of claim 1, wherein said second element comprises a receiver and a transmitter.

5. The system of claim 1, wherein said third element comprises a receiver and connecting means for connecting said third element to said hand-held electronic device.

6. The system of claim 1, wherein said third element comprises a transmitter.

7. The system of claim 1, wherein said transmitter of said first element and each receiver of said second element communicate via Bluetooth or IR-radiation.

8. The system of claim 1, wherein said second element comprises at least one transmitter and said third element comprises a receiver and wherein each transmitter of said second element and said receiver of said third element communicate via wireless connection selected from infrared and electromagnetic connection means or wired connection.

9. The system of claim 1, wherein said hand-held electronic device is selected from the group consisting of a conventional Smartphone, notebook and tablet computer.

10. The system of claim 1, wherein said hand-held electronic device is capable of displaying said position signal in a three dimensional illustration of the body.

11. The system of claim 1, wherein said first element comprises an acceleration sensor.

12. The system of claim 1, wherein said invasive device is an endotracheal tube, tracheostomy tube, stomach tube, oropharyngeal tube, cricothyrotomy tube, laryngeal mask, cardiac pacing wire or a venous, arterial or cardiac catheter.

13. The system of claim 1, wherein said body is an animal or human body.

14. The system of claim 1, wherein said position signal is visualized in real-time.

15. A method for employing the system of claim 1, comprising:
attaching a first element to a medical device to be inserted in a body,
attaching a second element to a predefined surface area of said body, and
connecting a third element to an electrical device comprising display means and being capable to receive and compute signals from the first and second elements,
wherein said third element is adapted to visualize said signals on said electronic device,
creating a positioning signal indicative for the position of said first element within the body, and visualizing the position signal on the electronic device,
preferably for avoiding Iatrogenic Traumatic Corporeal Disasters (ITCD).
